(19) United States
(12) Patent Application Publication
(10) Pub. No.: US 2009/0137437 A1
(43) Pub. Date: May 28, 2009

(54) Waxlike Ionomers

(75) Inventor: Gerd Hohner, Gersthofen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 12/313,632

(22) Filed: Nov. 21, 2008

(30) Foreign Application Priority Data
Nov. 23, 2007 (DE) 10 2007 056 533.1

(51) Int. Cl. C10M 129/28; C08L 51/06; C08L 33/02; C08L 91/06; C09D 11/10; C07C 57/03

(52) U.S. Cl. 508/459; 525/55; 525/78; 525/71; 524/556; 524/504; 562/598

(57) ABSTRACT

Through hydrolysis of reaction products of polyethylene or Fischer-Tropsch waxes and α,β-unsaturated carboxylic acids or their derivatives it is possible to gain access to innovative ionomer waxes which, in contrast to known ionomers of low molecular mass, exhibit low melt viscosities even at high degrees of hydrolysis. The ionomer waxes of the invention are suitable inter alia as components of pigment masterbatches, as plastics additives, and for application in polishes, paints, and printing inks.

The present invention is described in the German priority application No. 10 2007 056 533.1, filed Nov. 23, 2007, which is hereby incorporated by reference as is fully disclosed herein.

The present invention relates to waxlike ionomers (“ionomer waxes”) of low melt viscosity, prepared using reaction products of polyethylene waxes or paraffin waxes and α,β-unsaturated carboxylic acids or their derivatives.

Ionomers based on functionalized polyethylene structures, such as polyethylene structures containing carboxyl groups, are known. They are prepared by reacting, for example, polyethylene containing acid groups, prepared by copolymerizing ethylene and α,β-unsaturated carboxylic acids, in a neutralization or hydrolysis with metal oxides or metal hydroxides.

Polyethylene-based ionomers find use, for example, as additives for plastics, for instance as nucleators for influencing the crystallization behavior and the morphology, as (permanently active or migrating) antistats, and also as processing aids in shaping. They are additionally employed as pigment dispersants in the coloring of plastics with organic or inorganic pigments. From polyethylene ionomers it is possible, furthermore, to produce films for packaging, and coatings.

U.S. Pat. No. 3,264,272 describes ionomers obtained by partial hydrolysis of plasticlike copolymers of ethylene or 1-olefins and acrylic acid or methacrylic acid. In the course of the hydrolysis a drastic increase in viscosity is observed, evident from the decrease in the melt index value.

Known from EP 0 054 761 is the reaction of waxlike ethylene-acrylic acid copolymers with metal oxides or metal hydroxides. The viscosities of the raw materials used in the working examples are 500 or 650 mPa·s at 140° C.; in the course of the hydrolysis, the viscosity values increase to a multiple with increasing degree of hydrolysis. For degrees of hydrolysis of more than 50%, viscosity values are no longer reported.

EP 0 104 316 describes the production of saltlike products of low molecular mass by reaction of low molecular mass copolymers of ethylene and α,β-unsaturated carboxylic acids with oxides of group IIA of the Periodic Table of the Elements. The working examples use an ethylene-acrylic acid copolymer with an acrylic acid content of 3.9 mol % (trade name A-C 580) for the reaction. As reaction begins there is an increase in the melt viscosity.

Waxlike ionomers can be prepared in principle in a simple way, in a stirred tank process by stirred incorporation of suitable metal compounds into the melt of functionalized waxes. This procedure presupposes that the viscosity remains low enough, during the reaction, to ensure effective mixing of the reaction components and to avoid overloading of the stirring element. This aspect is especially significant when, in order to optimize the performance efficiency of the ionomers, it is necessary to set high degrees of neutralization or of hydrolysis, such as those close to 100%. The existing ionomers based on functionalized polyethylene waxes have extremely high melt viscosities or cannot be melted at relatively high degrees of hydrolysis. Their industrial production therefore necessitates special, laborious modes of operation. This is equally true of their application, where they are applied via the liquid melt state.

There is therefore a need for ionomers which are obtainable easily and economically and are characterized by a high degree of hydrolysis in tandem with a low melt viscosity.

Surprisingly it has been found that ionomers of this kind can be obtained by hydrolysis of waxes which have been functionalized by free-radical grafting with α,β-unsaturated carboxylic acids. More particularly, and unexpectedly, only a moderate increase in the melt viscosity, if any at all, is observed even in the case of complete hydrolysis, up to the maximum metal content.

The invention provides ionomer waxes having a melt viscosity as measured at 140° C. in the range from 5 to 30 000 mPa·s and a dropping or softening point in the range from 70 to 135° C., comprising Fischer-Tropsch waxes or polyethylene waxes which have been functionalized by free-radical grafting with α,β-unsaturated carboxylic acids or their derivatives and then hydrolyzed.

More particularly, in the case of the ionomer waxes of the invention, at least 30% of the functional groups present in the functionalized wax are hydrolyzed.

The ionomer waxes are prepared from functionalized waxes by reaction thereof with metal compounds, the functionalized waxes having been obtained by free-radical grafting of nonfunctionalized polyethylene waxes or Fischer-Tropsch waxes with α,β-unsaturated carboxylic acids or their derivatives.

Polyethylene waxes or polyethylenes containing carboxyl groups are accessible not only through oxidative degradation of polyethylenes but also through copolymerization of ethylene with unsaturated acids or by grafting of unsaturated acids onto ethylene polymers. Whereas, in the case of the copolymerization, the unsaturated carboxylic acid is incorporated into the main chain of the polymer molecule, the acid molecule appears as a side chain in the case of grafting. The grafting of acrylic acid onto polyethylene wax is known from the document JP 08-269 140, for example.

The waxes used as a graft base are understood, in contradistinction to plasticlike polyethylene, to be materials having low average degrees of polymerization or chain lengths. These qualities in turn imply low melt viscosities, which in the case of the waxes are typically in the range from about 5 to 30 000 mPa·s, as measured at 140° C., while in the case of the polyethylene plastics they are generally above 1000 Pa·s. The physical properties of the waxes differ significantly from those of the polyethylene plastics.

Polyethylene waxes can be prepared by thermal degradation of branched or unbranched polyethylene plastics or in a molecular enlargement process by direct polymerization of ethylene. Examples of suitable polymerization processes include free-radical techniques, in which ethylene is reacted at high pressures and temperatures to give waxes with a greater or lesser degree of branching; in addition, there are commonplace processes in which ethylene, where appropriate with addition of comonomers, is polymerized using organometallic catalysts, Ziegler or metallocene catalysts for example, to form unbranched or branched waxes. Corresponding methods of preparing ethylene homopolymer and copolymer waxes are described in, for example, Ullmann's Encyclopedia of Industrial Chemistry, 5th ed., Vol. A 28, Weinheim 1996 in sections 6.1.1./6.1.2. (high-pressure polymerization), section 6.1.2. (Ziegler-Natta polymerization, polymerization with metallocene catalysts), and section 6.1.4. (thermal degradation). The preparation of polyolefin waxes using metallocene catalysts is also described particularly by patents EP 0 321 852, EP 0 384 264, EP 0 571 882, and EP 0 890 584, for example.

Further compounds that can be used as polyethylene waxes are the shorter-chain fractions obtained as a byproduct in the production of polyethylene plastic by the Ziegler process, provided these fractions conform to the pattern of properties of polyethylene waxes.

Suitable polyethylene waxes are not only homopolymers of ethylene but also its copolymers with one or more 1-olefin(s) R—CH═CH2 in which R is a straight-chain or branched alkyl radical having 1 to 20 carbon atoms. The comonomer content here may be between 0.1% and 49% by weight.

As a graft base it is also possible, furthermore, to use waxlike hydrocarbons obtained by the Fischer-Tropsch process, i.e., by catalytic reaction of carbon oxide with hydrogen (“Fischer-Tropsch paraffins”; on this point see Ullmann's Encyclopedia of Industrial Chemistry, 5th ed., Vol. A 28, Weinheim 1996, chapter 5). These waxes have low melt viscosities (below 20 mPa·s as measured at 140° C.).

Preferred starting materials for the grafting are polyethylene waxes obtained by direct polymerization, more preferably those prepared using Ziegler catalysts or metallocene catalysts. Equally preferred are Fischer-Tropsch paraffins.

Graft monomers contemplated include both monobasic and polybasic α,β-unsaturated carboxylic acids. Examples of suitable monobasic acids are acrylic acid, methacrylic acid, ethacrylic acid or crotonic acid. Examples of polybasic acids are maleic acid or fumaric acid. The acids may be used both individually and in plurality as a mixture. Besides the free acids it is also possible to use their derivatives, provided the resulting graft products can subsequently be converted into ionomers. Such derivatives include, for example, esters, examples being esters of acrylic acid such as methyl acrylate, or anhydrides, an example being maleic anhydride. Preferred graft components are acrylic acid and methacrylic acid, with acrylic acid being particularly preferred.

For the graft reaction the α,β-unsaturated carboxylic acids are used in amounts of 0.1% to 60% by weight, based on nonfunctionalized wax employed. The grafting is generally initiated using a free-radical initiator, preferably an organic peroxo compound, such as an alkyl hydroperoxide, a dialkyl or diaryl peroxide or a peroxo ester, for example. The graft reaction may be carried out in solution or in the melt at temperatures adapted to the decomposition characteristics of the peroxide. Preference is given to reaction in the melt.

The polyethylene ionomers are prepared in principle by treating the polyethylene wax obtained by grafting with α,β-unsaturated carboxylic acids or their derivatives, in the liquid melt state or in solution, preferably in the melt, with a metal compound which converts some or all of the acid and/or acid-equivalent functions that are present in the wax into carboxylate functions. The metal compounds used comprise metals preferably of groups IA, IIA, IIIA, IB, IIB, and VIIIB of the Periodic Table of the Elements, more preferably alkali metals and alkaline earth metals, and also zinc. Metal compounds contemplated are generally those which can be converted with acid or acid-equivalent functions into metal carboxylates, examples being hydroxides or oxides. It is also possible to use metal compounds with a salt character, more particularly salts of volatile acids. Preference, however, is given to hydroxides and/or oxides. Examples of metal compounds include sodium or potassium hydroxide, calcium and magnesium oxide or hydroxide, aluminum hydroxide, and also zinc oxide or hydroxide. In one preferred preparation process the melt of the acid wax is introduced as an initial charge and the metal compound, as it is or in solution or dispersion in water, is introduced into the wax melt with stirring. Added water and any water of reaction formed can be removed during the reaction or subsequently by distillation, under atmospheric pressure or subatmospheric pressure, and/or by means of a gas stream, preferably an inert gas stream. The reaction may in principle take place batchwise or continuously.

The proportion of metal compound employed and functionalized polyethylene wax employed is chosen such that a degree of hydrolysis of at least 30% is reached, preferably at least 50%, more preferably at least 70%. Very particular preference is given to degrees of hydrolysis of between 80% and 100%. The degree of hydrolysis indicates the percentage stoichiometric fraction of the acid or acid-equivalent groups originally present that have been converted into carboxylate.

The extent of the formation of carboxylate can be monitored, for example, by determination of the acid number of by means of IR spectroscopy during the reaction, and/or can be ascertained on the end product.

The ionomer waxes of the invention have melt viscosities as measured at 140° C. of less than 30 000, preferably less than 10 000, more preferably less than 5000, with especial preference less than 1000 mPa·s. Their dropping points lie between 70 and 135° C., preferably between 90 and 130° C.

The ionomer waxes can be converted into powders by spraying or grinding and can also be used in that form if advantageous or necessary from a performance standpoint. On account of their high hardness and brittleness they are especially suitable for grinding, on jet mills or mechanical mills, for example. Where brittleness is sufficient, extremely small particle sizes can be achieved, as for example d50 values of <8 μm. The d50 value is a statement to the effect that 50% of the particles have a size below the respective value. The ionomer waxes may also be used in an arbitrarily coarser form, in the form of granules, for example.

The ionomer waxes can be employed, for example, as pigment dispersants in the coloring of thermoplastics with organic or inorganic pigments, as nucleators for influencing the crystallization behavior and the morphology of thermoplastics, and also as (permanently active or migrating) antistatic additives. They are suitable as lubricants and release agents for plastics processing, and can be processed to aqueous or solventborne dispersions for polishing or industrial applications. They can be used, furthermore, as matting additives and abrasion protection additives for coating materials, and for optimizing the mechanical stability and slip properties of printing-ink films.

### EXAMPLES

The invention is illustrated with reference to the following working examples.

The melt viscosities were determined in accordance with DIN 51562 using a rotary viscometer; the dropping and softening points were determined in accordance with DIN 51801/2 and DIN EN 1427, respectively; and the acid numbers were determined in accordance with DIN 53402. IR spectroscopy measurements were carried out using the Vector 22 instrument (Bruker).

### Examples 1-17

Preparation of Waxes Grafted with α,β-Unsaturated Acids (“Wax Acids”)

2500 g of each of the nonfunctional waxes listed in table 1, column 2, were melted in a nitrogen-blanketed glass apparatus equipped with stirrer mechanism, internal thermometer, and distillation bridge. At a temperature of 140° C., with stirring, the α,β-unsaturated acid listed in column 3 was metered in continuously from a metering funnel over the course of 3 h; at the same time, from a second dropping funnel, the continuous addition took place of 25.0 g of di-tert-butyl peroxide. After the end of metering, the temperature was raised to 160° C. and reaction was allowed to continue for 1 h. Subsequently a vacuum (approximately 30 mbar) was applied and the volatile fractions were removed by distillation. After about 30 min, nitrogen was admitted to let the pressure down to atmospheric pressure. Melt viscosity and acid number of the resulting wax acids are listed in columns 5 and 6.

The following waxes were employed as starting materials:

Licocene® PE 4201, Licocene® PE 5301: ethylene homopolymer waxes produced using metallocene catalysts, commercial product of Clariant Produkte (Deutschland) GmbH;

Licowax® PE 130: ethylene homopolymer wax produced using a Ziegler catalyst, commercial product of Clariant Produkte (Deutschland) GmbH;

Sasolwax H1: Fischer-Tropsch paraffin, commercial product of Sasolwax.

Reaction of the Wax Acids to Ionomer Waxes

2500 g of each of the functionalized waxes obtained by grafting were melted under nitrogen blanketing in a glass apparatus equipped with stirrer mechanism, internal thermometer, and distillation bridge. At a temperature of 160° C., with effective stirring, the alkali metal hydroxide or alkaline earth metal hydroxide specified in column 7 of table 1, in powder form, was introduced over the course of approximately 15 min in portions, in the equivalent ratio specified in column 8. Stirring was continued at 165° C. for 30 min. Subsequently, for drying, vacuum was applied until the melt was free of bubbles. The properties of the resultant ionomer waxes are listed in table 1.

TABLE 1

| 1 | 2 | 3 | 4 | 5 | 6 | 7 | 8 | 9 | 10 | 11 |
|---|---|---|---|---|---|---|---|---|---|---|
| | Preparation/ | | | | Preparation of | | | Properties of | | |
| | properties of wax acid | | | | ionomer wax | | | ionomer wax | | |
| | | | Amount of acid | | Acid | | Base/wax | | Acid | |
| | | | component | Viscosity/ | number | | acid | Viscosity/ | number | Dropping |
| | | Acid | used | 140° C. | (mg | | equivalent | 140° C. | (mg | point |
| Ex. | Raw wax material | component | (% by weight)¹⁾ | (mPa · s) | KOH/g) | Base | ratio | (mPa · s) | KOH/g) | (° C.) |
| 1 | Licocene ® PE 4201 | Acrylic acid | 7.5 | 106 | 51 | KOH | 1.00 | 120 | 2 | 120 |
| 2 | Licocene ® PE 4201 | Acrylic acid | 10 | 107 | 67 | KOH | 0.95 | 233 | 2 | 120 |
| 3 | Licocene ® PE 4201 | Acrylic acid | 15 | 93 | 95 | KOH | 0.95 | 174 | 3 | 121 |
| 4 | Licocene ® PE 4201 | Acrylic acid | 20 | 130 | 128 | KOH | 0.95 | 217 | 7 | 122 |
| 5 | Licocene ® PE 4201 | Acrylic acid | 10 | 107 | 67 | NaOH | 0.95 | 159 | 3 | 119 |
| 6 | Licocene ® PE 4201 | Acrylic acid | 20 | 104 | 124 | NaOH | 0.95 | 222 | 3 | 120 |
| 7 | Licocene ® PE 4201 | Acrylic acid | 10 | 107 | 67 | LiOH | 0.95 | 124 | 9 | 119 |
| 8 | Licocene ® PE 4201 | Acrylic acid | 10 | 107 | 67 | Ca(OH)2 | 0.95 | 155 | 6 | 121 |
| 9 | Licocene ® PE 4201 | Acrylic acid | 10 | 107 | 67 | ZnO | 0.95 | 134 | — | 121 |
| 10 | Licocene ® PE 4201 | Methacrylic | 10 | 108 | 50 | KOH | 0.95 | 109 | 4 | 123 |
| | | acid | | | | | | | | |
| 11 | Licowax ® PE 130 | Acrylic acid | 10 | 908 | 63 | KOH | 0.95 | 1289 | 4 | 123 |
| 12 | Licowax ® PE 130 | Methacrylic | 10 | 852 | 57 | KOH | 0.95 | 920 | 2 | 124 |
| | | acid | | | | | | | | |
| 13 | Licocene ® PE 5301 | Acrylic acid | 10 | 397 | 65 | KOH | 0.95 | 1025 | 7 | 123 |
| 14 | Licocene ® PE 5301 | Methacrylic | 10 | 660 | 51 | KOH | 0.95 | 710 | 6 | 123 |
| | | acid | | | | | | | | |
| 15 | Ethylene-propylene | Acrylic acid | 10 | 410 | 65 | KOH | 0.95 | 590 | 3 | 122 |
| | copolymer wax²⁾ | | | | | | | | | |
| 16 | Sasolwax H1 | Acrylic acid | 10 | 11 | 68 | KOH | 0.95 | 11 | 4 | 110 |
| 17 | Sasolwax H1 | Acrylic acid | 20 | 14 | 118 | KOH | 0.95 | 17 | 8 | 111 |

¹⁾based on raw wax material employed
²⁾produced with a metallocene catalyst as per EPS 0571882, example 11, melt viscosity/140° C. 260 mPa · s

In accordance with their high degree of hydrolysis, the ionomer waxes prepared have low acid numbers of below 10 mg KOH/g, predominantly below 5 mg KOH/g. In the IR spectra there are minor absorption signals or none in the region of the acid carbonyl group (approximately 1700-1710 cm-1), but intense carboxylate bands can be made out in the 1610-1550 or 1420-1300 cm-1 range.

### Examples C1 and C2 (Comparative Examples)

Employing the procedure described above for the reaction of wax acid to ionomer wax, the wax A-C 540 (commercial product of Honeywell), prepared by copolymerization of ethylene with acrylic acid, was reacted with potassium hydroxide in a KOH:wax acid equivalent ratio of 0.375. The feedstock wax had an acid number of 41 mg KOH/g and a melt viscosity as measured at 140° C. of 486 mPa·s; the resultant ionomer wax gave an acid number of 26 mg KOH/g and a melt viscosity at 140° C. of 3390 mPa·s. When 0.50 equivalent of KOH was used, based on wax acid employed, the viscosity of the melt after a short time was so high that it was necessary to shut off the stirrer.

An analogous experiment with the wax A-C 580 (likewise a copolymer of ethylene and acrylic acid, commercial product from Honeywell, acid number 76 mg KOH/g, viscosity at 140° C., 672 mPa·s), carried out using 0.375 equivalent of potassium hydroxide per equivalent of wax acid, gave ionomer product having an acid number of 45 mg KOH/g and a viscosity at 140° C. of 4634 mPa·s.

The comparative experiments show that the hydrolysis of wax acids prepared by copolymerization from ethylene and acrylic acid leads, even at comparatively low degrees of hydrolysis, to high-viscosity ionomer products which can no longer be managed in typical stirring processes.

1. An ionomer wax having a melt viscosity measured at 140° C. of less than 30 000 mPas and a dropping or softening point in the range from 70 to 135° C., comprising one or more Fischer-Tropsch waxes or one more polyethylene waxes, wherein the one or more Fischer-Tropsch waxes or one more Polyethylene waxes are functionalized by free-radical grafting with α,β-unsaturated carboxylic acids or their derivatives and then hydrolyzed.

2. The ionomer wax as claimed in claim 1, wherein at least 30% of the functional groups present in the one or more Fischer-Tropsch waxes or one more polyethylene waxes functionalized by free-radical grafting with α,β-unsaturated carboxylic acids or their derivatives are hydrolyzed.

3. A process for preparing an ionomer wax having a melt viscosity of less than 30 000 mPas comprising the step of reacting one or more functionalized polyethylene waxes with one or more metal compounds, wherein the one or more functionalized waxes are obtained by free-radical grafting of nonfunctionalized polyethylene waxes or Fischer-Tropsch waxes with α,β-unsaturated carboxylic acids or their derivatives.

4. The process as claimed in claim 3, wherein the nonfunctionalized polyethylene waxes have been prepared by polymerizing using Ziegler catalysts or metallocene catalysts.

5. The process as claimed in claim 3, wherein the nonfunctionalized polyethylene waxes are homopolymers of ethylene or copolymers of ethylene with one or more 1-olefin(s) of the formula R—CH═CH₂ in which R is a straight-chain or branched alkyl radical having 1 to 20 carbon atoms.

6. The process as claimed in claim 3, wherein the nonfunctionalized polyethylene waxes are copolymers of ethylene containing 0.1% to 49% by weight of one or more 1-olefin(s) of the formula R—CH═CH₂ in which R is a straight-chain or branched alkyl radical having 1 to 20 C carbon atoms.

7. The process as claimed in claim 3, wherein the one or more metal compounds comprise metals from groups IA, IIA, IIIA, IB, IIB or VIIIB of the Periodic Table of the Elements.

8. The process as claimed in claim 3, wherein the one or more metal compounds are metal oxide, metal hydroxide compounds or a mixture thereof.

9. The process as claimed in claim 3, wherein the proportion of the one or more metal compounds employed and one or more functionalized polyethylene waxes employed is chosen such that a degree of hydrolysis of at least 30% is reached.

10. An ionomer wax obtained by a process as claimed in claim 3.

11. A pigment dispersant, as a nucleator, a lubricant or a release agent in plastics processing comprising an ionomer wax as claimed in claim 1.

12. The use as claimed in claim 11, wherein the ionomer wax is used as a powder having d50 values <8 μm.

13. The process as claimed in claim 3, wherein the metal compounds comprise metals selected from the group consisting of alkali metals, alkaline earth metals, zinc and aluminum.

14. A matting additive or abrasion protection additive for coating materials comprising an ionomer wax as claimed in claim 1.

15. An additive in the production of printing-ink films comprising an ionomer wax as claimed in claim 1.
